(19) United States
(12) Patent Application Publication
(10) Pub. No.: US 2018/0296145 A1
(43) Pub. Date: Oct. 18, 2018

(54) SYSTEM AND METHOD FOR DETECTION OF NEUROTRANSMITTERS AND PROTEINS IN THE CARDIAC SYSTEM

(71) Applicants: THE REGENTS OF THE UNIVERSITY OF CALIFORNIA, Oakland, CA (US); CASE WESTERN RESERVE UNIVERSITY, Cleveland, OH (US)

(72) Inventors: Kalyanam Shivkumar, Los Angeles, CA (US); Jeffrey Laurence Ardell, Los Angeles, CA (US); Corey Smith, Cleveland, OH (US)

(21) Appl. No.: 15/910,633

(22) Filed: Mar. 2, 2018

Related U.S. Application Data
(60) Provisional application No. 62/485,880, filed on Apr. 14, 2017.
(60) Provisional application No. 62/570,237, filed on Oct. 10, 2017.

(51) Int. Cl. A61B 5/1473; A61K 49/00; C07K 16/26; A61B 5/145; A61B 5/00; A61B 5/1495

(52) U.S. Cl. CPC A61B 5/14735; A61K 49/0004; C07K 16/26; A61B 5/14546; A61B 2562/028; A61B 5/6876; A61B 5/6877; A61B 5/1495; A61B 5/6869

(57) ABSTRACT

The present invention provides a device and methods of use related to the use of electrodes to detect the presence and abundance of various biochemical compounds of interest with high spatial and temporal resolution.

### CROSS-REFERENCE TO RELATED APPLICATIONS

This application claims the benefit of priority under 35 U.S.C. § 119(e) to U.S. Provisional Patent Application No. 62/485,880, filed Apr. 14, 2017, and to U.S. Provisional Patent Application No. 62/570,237, filed Oct. 10, 2017, the contents of each of which are incorporated by reference herein in their entirety.

### STATEMENT REGARDING FEDERALLY SPONSORED RESEARCH OR DEVELOPMENT

This invention was made with government support under 1RO1GM102191 awarded by the National Institutes of Health. The government has certain rights in the invention.

### BACKGROUND OF THE INVENTION

Catecholamines and other neurotransmitters are produced by central neurons, peripheral autonomic sympathetic neurons and neuroendocrine chromaffin cells of the adrenal gland and serve a variety of functions in normal physiology and pathophysiology. When released in the central and peripheral nervous systems they can function as neuromediators/neuromodulators and when released in the blood circulation, they can function as hormones. Currently, there is no means by which to directly measure the concentration of catecholamine or other neurotransmitters in near real-time in the heart under normal conditions or in response to stressors. The current state of the art in monitoring cardiac autonomic function or dysfunction uses blood tests or tissue biopsy, which are less accurate and carry a higher risk of infection or scarring tissue.

Thus, there is a need in the art for a system and method for precise detection and monitoring of neurotransmitters in the heart to evaluate cardiac function or dysfunction. The present invention satisfies this unmet need.

### SUMMARY OF THE INVENTION

In one aspect the present method provides a method of detecting a biochemical compound. In one embodiment, the method comprises the steps of: inserting one or more electrodes in one or more locations selected from the group consisting of: the heart, neural structure, and peripheral blood vessel; applying a voltage scan to the electrode; and detecting a current indicative of the presence and abundance of the compound. In certain embodiments, the method is used to monitor cardiac autonomic function or dysfunction. In certain embodiments, the method provides for detection of regional differences of the biochemical compound.

In one embodiment, the one or more electrodes are placed into the myocardium. In one embodiment, the one or more electrodes are placed in one or more locations selected from the group consisting of: a coronary sinus of the heart, a great vein of the heart, vena cava, left ventricle, aorta, right ventricle, right atria, left atria, pulmonary veins, pulmonary artery, stellate ganglia, dorsal root ganglia, epicardial fat pad, and pericardial fat pad. In one embodiment, the one or more electrodes are inserted via epicardial or vascular access.

In one embodiment, the compound is at least one catecholamine selected from the group consisting of norepinephrine and epinephrine.

In one embodiment, at least one electrode is an electrode selected from the group consisting of: wire electrodes, microwire electrodes, needle electrodes, plunge electrodes, penetrating electrodes, patch electrodes, single shank electrodes, 2D shank electrodes, 3D shank electrodes, and multi-electrode arrays.

In one embodiment, the voltage scan is a fast scanning cyclic voltammetry (FSCV) voltage scan. In one embodiment, the FSCV voltage scan comprises a waveform selected from the group consisting of: a sawtooth pattern and sinusoidal pattern.

In one embodiment, the method comprises detecting the oxidation current of the compound. In one embodiment, the method comprises constructing a voltammogram from the detected current, thereby identifying the compound. In one embodiment, the method comprises quantifying the abundance of the compound by plotting the peak current on a calibration curve.

In one embodiment, the presence and abundance of the biochemical compound is assessed in response to one or more cardiac stressors.

In one embodiment, a plurality of electrodes are placed at a plurality of locations within and around the heart to assess regional differences in the abundance of the biochemical compound.

In one aspect, the present invention provides for a method for detecting a biochemical compound comprising the steps of: inserting one or more electrodes in one or more locations selected from the group consisting of: the heart, neural structure, and peripheral blood vessel, wherein at least one electrode comprises a receptor molecule that specifically binds the biochemical compound; and detecting a change in the capacitance of the electrode thereby indicating the presence of the biochemical compound.

In one embodiment, the biochemical compound is a protein or peptide that specifically binds to the receptor molecule.

In one embodiment, the level of the compound is detected in at least one ganglia selected from the group consisting of intrathoracic ganglia, stellate ganglia, autonomic ganglia, nodose ganglia, dorsal root ganglia and petrosal ganglia. In one embodiment, one or more electrodes are placed in a peripheral artery or peripheral vein.

In one aspect, the present invention provides a biochemical compound detection device, comprising: a controller, comprising a potentiostat; a reference electrode communicatively connected to the controller; and one or more measurement electrodes communicatively connected to the controller; wherein the controller is configured to apply an electric potential across the reference electrode and the one or more measurement electrodes, and to measure the current passing through the one or more measurement electrodes over time; and wherein the reference electrode and one or more measurement electrodes are configured to measure the presence and concentration of one or more biochemical compounds.

In one embodiment, the device comprises a ground electrode, wherein the controller is configured to apply an electric potential across the reference electrode and the ground electrode.

In one embodiment, at least one measurement electrode comprises a receptor molecule that specifically binds to a biochemical compound.

In one embodiment, the device further comprises a semi-permeable membrane applied to a portion of an electrode selected from the group consisting of the reference electrode, the measurement electrode, and the ground electrode.

In one embodiment, at least one of the electrodes selected from the group consisting of the measurement electrode and the reference electrode are made of platinum. In one embodiment, at least one of the electrodes selected from the group consisting of the measurement electrode and the reference electrode are made from carbon fiber.

In one embodiment, the controller further comprises a voltage clamp, configured to maintain a substantially constant voltage across two or more electrodes.

### BRIEF DESCRIPTION OF THE DRAWINGS

FIG. 1 depicts a schematic of an exemplary use of voltammetry for diagnostic and therapeutic use.

FIG. 2 depicts a schematic of an exemplary embodiment of a method of the invention as described herein.

FIG. 3 depicts an exemplary graphic user interface (GUI) for the control of parameters for FSCV acquisition. The interface was written in the IGOR Pro environment (Wavemetrics, Inc.).

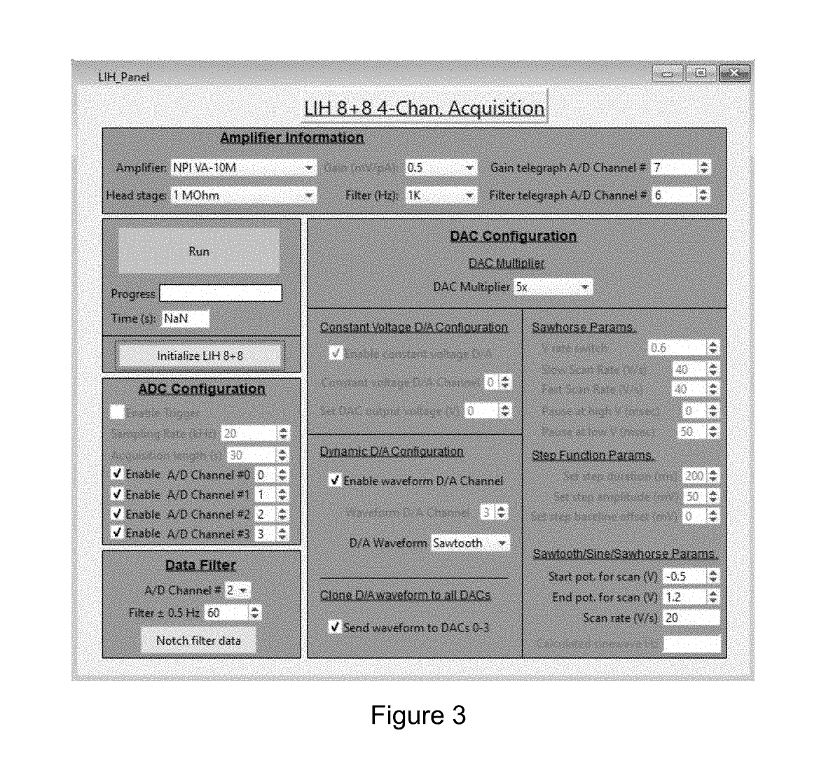

FIG. 4 depicts the voltage clamp circuit for the invention as described herein.

FIG. 5A through FIG. 5D depict exemplary elements of FSCV. FIG. 5A depicts an exemplary voltage scan delivered to an electrode. FIG. 5B depicts exemplary raw FSCV currents. Current versus time is recorded through a platinum electrode. A two second current is shown. FIG. 5C depicts a voltammogram demonstrating the current at baseline and in the presence of epinephrine. FIG. 5D depicts the oxidation current of epinephrine, obtained by subtracting out the background current.

FIG. 6A and FIG. 6B depict exemplary FSCV recordings for the detection of norepinephrine (NE) (FIG. 6A) and epinephrine (Epi) (FIG. 6B) at known concentrations. The depicted results indicate that Norepinephrine has a unique current versus voltage profile from that of Epinephrine, indicating the signal from these two catecholamines is separable and distinct.

FIG. 7 depicts exemplary calibration curves for quantifying the concentration of norepinephrine (left) and epinephrine (right) from a measured current in picoamperes (pA) using FSCV.

FIG. 8 depicts a schematic and exemplary results of real time interstitial cardiac catecholamine detection in response to left anterior descending coronary artery occlusion.

FIG. 9 depicts a kymograph illustrating the oxidation potential as a function of voltage and time, where the presence of norepinephrine is detected prior to, during and following manual coronary arterial occlusion protocol, reflecting an increased oxidation current characteristic for norepinephrine.

FIG. 10 depicts the results from experiments where FSCV was used to detect the presence of norepinephrine from 4 electrodes placed at 4 different regions of the heart relative to induced ischemic zone during LAD occlusion, demonstrating the ability to measure FSCV at high time resolution in sub-regions of the heart.

FIG. 11 illustrates a schematic of a functionalized electrode modeled as a resistance-capacitance (RC) circuit wherein a ligand selectively binds receptors linked to the tip of an exemplary electrode thereby altering the capacitance and thus impedance of the electrode. The amplitude of the change in current detected indicates selective binding between ligand and receptor.

FIG. 12 depicts an exemplary calibration curve of enkephalin concentration.

FIG. 13 depicts results from experiments using impedance measurements to indicate peptide detection following splenic nerve stimulation at the adrenal gland.

### DETAILED DESCRIPTION

The present invention provides a system, device, and method for detecting biomolecules in the heart to assess and monitor cardiac function or dysfunction. For example, in certain aspects, the invention relates to the detection of neurotransmitters, including, but not limited to catechlamines, such as epinephrine and norepinephrine. In some aspects, the invention relates to the detection of proteins. For example, in certain embodiments, the invention relates to the detection of neurotransmitters and/or proteins that are released by one or more cells or by the autonomic nervous system. In certain embodiments, the method relates to the detection of a cardiac event by detecting and monitoring the presence and/or abundance of neurotransmitters and/or proteins in the heart.

Catecholamines are produced and released by chromaffin cells and serve a variety of functions in the heart under normal physiological and pathophysiological conditions. For example, when released in the central and peripheral nervous systems, catecholamines function as neuromediators/neuromodulators, and when released in the blood circulation, catecholamines function as hormones. The ability to detect expression and concentration of such compounds offers insight into the function or dysfunction of the heart or cardiac nervous system. The present invention allows for the measurement of neurotransmitters and proteins with high temporal and spatial resolution. The presently described device, system, and method can be used to monitor cardiac autonomic function or dysfunction by measuring and monitoring the presence, abundance, and location of neurotransmitters and proteins in the heart.

The ability to measure such compounds in response to stimuli in the heart provides great insight into normal and abnormal function of the heart and the role that compounds such as catecholamines play in pathophysiology. The present invention provides a device and methods for detecting catecholamines in addition to other neuromodulators and hormones in order to better determine proper function of effector organs. The ability to detect expression and concentration of such compounds can offer insight into proper function of target organs of such compounds, including the heart.

The ability to measure regional differences in catecholamines in addition to other neuromodulators and hormones provides greater insights into normal and abnormal function of the neural-heart interface that can be predictive of adverse outcomes, including potential for arrhythmias and heart failure. The ability to measure regional differences in catecholamines in addition to other neuromodulators and hormones provides a methodology to rapidly assess efficacy to therapeutic interventions. The ability to measure regional differences in the vascular compartment for catecholamines in addition to other neuromodulators and hormones provides greater insight into relevant biomarkers indicative of susceptibility to cardiac pathology and the progression of the cardiovascular disease process.

Current strategies for detecting catecholamines in the cardiac setting include microdialysis of the interstitial fluid, followed by off-line detection by high performance liquid chromatography and electrochemical detection. This approach has a limited temporal resolution of minutes, an analytic time requirement of minutes to hours and are accomplished in a diagnostic lab setting. The process described herein has a temporal resolution on the milliseconds time scale, an analytic time requirement of minutes to near real-time and can be accomplished at the bedside. Moreover, application of the process described herein may be accomplished through a minimally invasive catheter deployment, a characteristic not available to the current methodologies.

### Definitions

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, exemplary methods and materials are described.

As used herein, each of the following terms has the meaning associated with it in this section.

The articles “a” and “an” are used herein to refer to one or to more than one (i.e., to at least one) of the grammatical object of the article. By way of example, “an element” means one element or more than one element.

“About” as used herein when referring to a measurable value such as an amount, a temporal duration, and the like, is meant to encompass variations of ±20%, ±10%, ±5%, ±1%, or ±0.1% from the specified value, as such variations are appropriate to perform the disclosed methods.

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

In some aspects of the present invention, software executing the instructions provided herein may be stored on a non-transitory computer-readable medium, wherein the software performs some or all of the steps of the present invention when executed on a processor.

Aspects of the invention relate to algorithms executed in computer software. Though certain embodiments may be described as written in particular programming languages, or executed on particular operating systems or computing platforms, it is understood that the system and method of the present invention is not limited to any particular computing language, platform, or combination thereof. Software executing the algorithms described herein may be written in any programming language known in the art, compiled or interpreted, including but not limited to C, C++, C#, Objective-C, Java, JavaScript, Python, PHP, Perl, Ruby, or Visual Basic. It is further understood that elements of the present invention may be executed on any acceptable computing platform, including but not limited to a server, a cloud instance, a workstation, a thin client, a mobile device, an embedded microcontroller, a television, or any other suitable computing device known in the art.

Parts of this invention are described as software running on a computing device. Though software described herein may be disclosed as operating on one particular computing device (e.g. a dedicated server or a workstation), it is understood in the art that software is intrinsically portable and that most software running on a dedicated server may also be run, for the purposes of the present invention, on any of a wide range of devices including desktop or mobile devices, laptops, tablets, smartphones, watches, wearable electronics or other wireless digital/cellular phones, televisions, cloud instances, embedded microcontrollers, thin client devices, or any other suitable computing device known in the art.

Similarly, parts of this invention are described as communicating over a variety of wireless or wired computer networks. For the purposes of this invention, the words “network”, “networked”, and “networking” are understood to encompass wired Ethernet, fiber optic connections, wireless connections including any of the various 802.11 standards, cellular WAN infrastructures such as 3G or 4G/LTE networks, Bluetooth®, Bluetooth® Low Energy (BLE) or Zigbee® communication links, or any other method by which one electronic device is capable of communicating with another. In some embodiments, elements of the networked portion of the invention may be implemented over a Virtual Private Network (VPN).

### Description

The present invention relates to a device, system, and method for real-time detection of neurotransmitters and proteins in the heart.

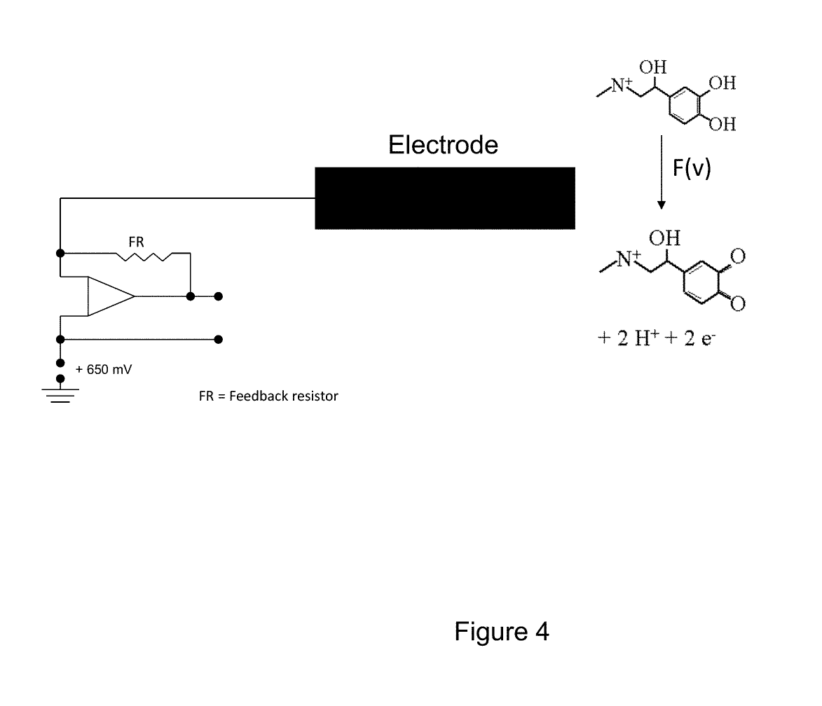

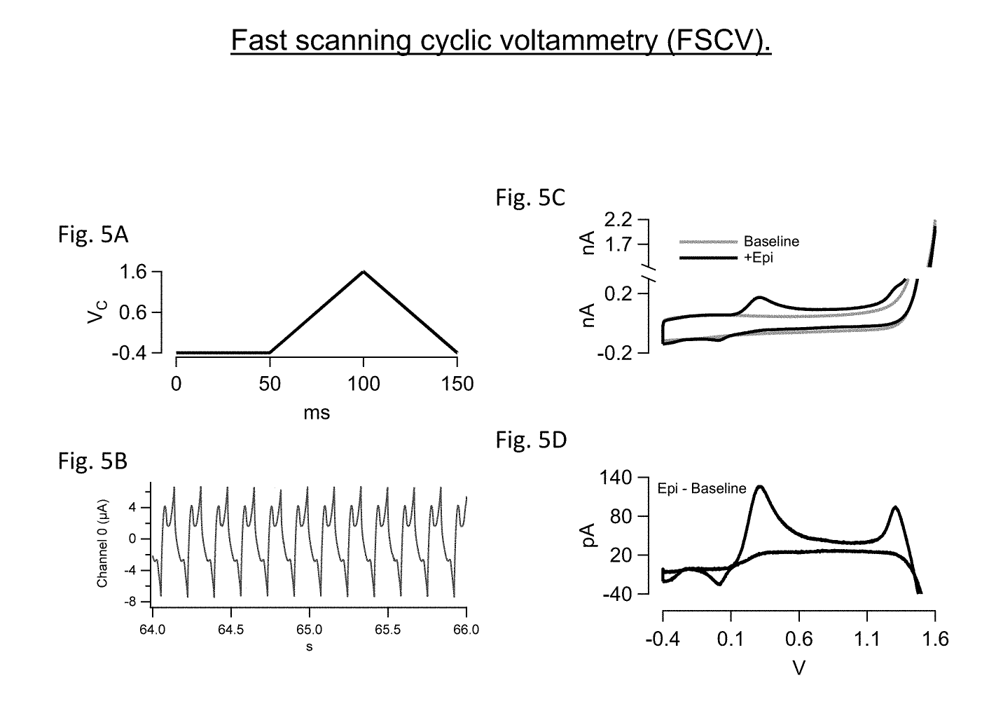

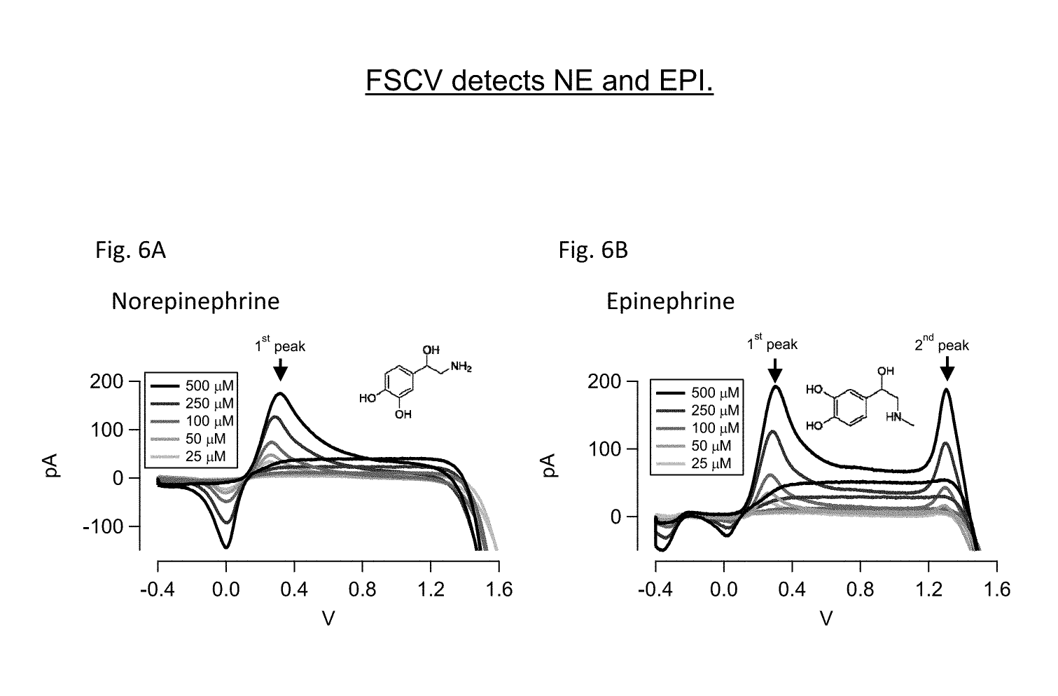

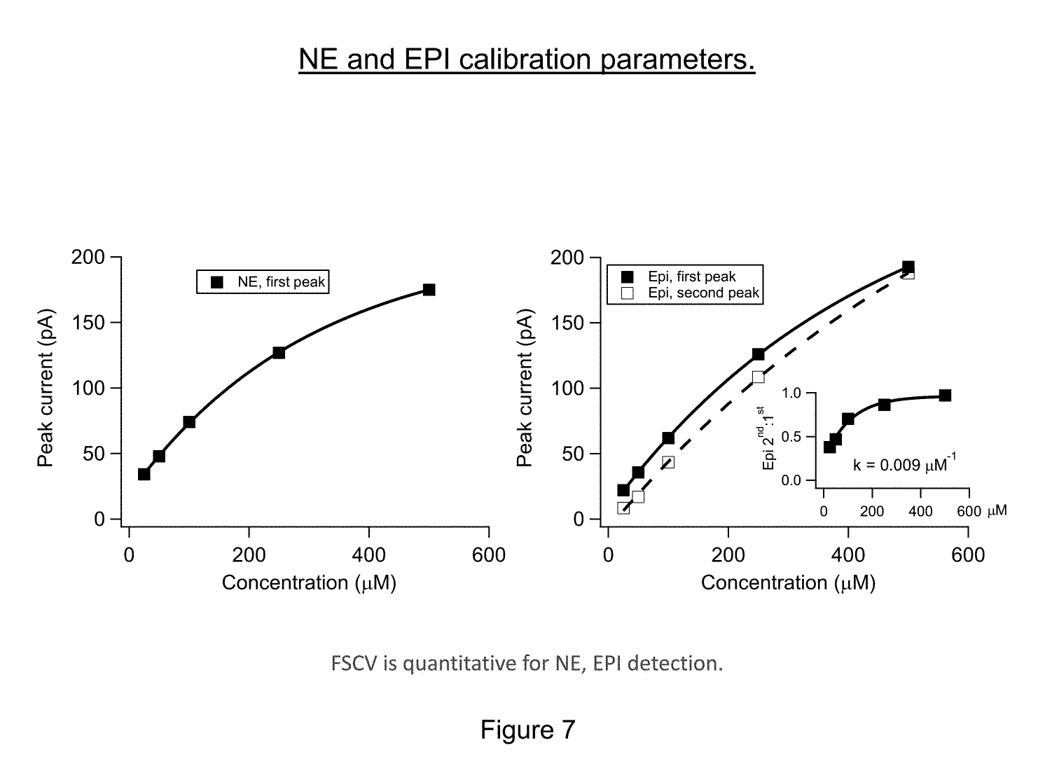

In one aspect, the invention relates to the use of voltammetry to measure the presence and abundance of one or more neurotransmitters, including but not limited to epinephrine and norepinephrine. In a specific embodiment, the invention relates to the use of fast scanning cyclic voltammetry (FSCV), which relates to a technique where the voltage of an implanted electrode is quickly and cyclically increased and then decreased, typically in a triangular or sinusoidal wave pattern. The charge imparted to the electrode tip generates an electric field, which causes oxidation and reduction reactions of compounds in the vicinity of the electrode tip. The reactions, in turn, induce a measurable current in the electrode through a voltage clamp circuit, for example a voltage clamp circuit as depicted in FIG. 4. Subtraction of the background current from the total current measured produces a voltage versus current plot (i.e. a voltammogram) of the current induced by the oxidation-reduction reactions as depicted in FIG. 5C-FIG. 5D. For example, the characteristic voltammogram produced by the oxidation and reduction of norepinephrine at the electrode tip is shown in FIG. 6A, while the characteristic voltammogram produced by the oxidation and reduction of epinephrine at the electrode tip is shown in FIG. 6B. The amplitude of the current at the characteristic peak is correlated with the concentration of the compound present at the vicinity of the electrode tip. Higher concentrations of compounds result in more oxidation and reduction reactions, which in turn induce a higher total current as shown in FIGS. 6 and 7. However, the present invention is not limited to the use of FSCV, but rather encompasses the use of any type of voltammetry that induces current from the oxidation and/or reduction of biochemical species in the vicinity of the electrode tip. Other exemplary forms of voltammetry include, but are not limited to, potential step voltammetry, linear sweep voltammetry cyclic voltammetry, square wave voltammetry, staircase voltammetry, anodic or cathodic stripping voltammetry, adsorptive stripping voltammetry, alternating current voltammetry, rotated electrode voltammetry, normal or differential pulse voltammetry, chronoamperometry, and chronocoulometry.

In one embodiment, the invention relates to the use of capacitive immunosensors to detect the presence and abundance of a biochemical compound, such as a protein, peptide, nucleic acid, hormone, or the like. For, example, in certain embodiments, the capacitive immunosensors comprise an electrode functionalized with a capture agent, such as an antibody or probe, that specifically binds the biochemical compound. Binding of the compound to the capture agent results in a change in the capacitance of the electrode. Thus, a detected change in capacitance is indicative of the presence and abundance of the biochemical compound of interest.

The present invention provides a device for detecting the presence and abundance of one or more biochemical compounds, including, but not limited to, neurotransmitters, such as epinephrine and norepinephrine, proteins, peptides, nucleic acids, and the like. In one embodiment, the device comprises one or more electrodes configured for implantation into the heart of a subject. The one or more electrodes may comprise any suitable electrode suitable for delivering and measuring a potential. For example, the electrode may comprise a conducting metal, including but not limited to alloys such as indium tin oxide, conductive carbon, or noble metals such as gold, silver, palladium or platinum. Suitable electrodes include, but are not limited to, needle electrodes, plunge electrodes, penetrating electrodes, patch electrodes, single shank electrodes, 2D shank electrodes, 3D shank electrodes, multi-electrode arrays, wire electrodes, microwire electrodes, or the like. In certain embodiments, the device comprises a microelectrode array comprising a plurality of electrode tips suitable for implantation into the target tissue or suitable for placement within the vascular space.

In certain embodiments, the one or more electrodes comprise a wire, microwire, or collection of wires or microwires. In certain embodiments, the electrode comprises a wire electrode having a diameter in the range of about 1 μm to about 5 mm. In one embodiment, the electrode comprises a wire electrode having a diameter in the range of about 10 μm to about 1 mm. In one embodiment, the electrode comprises a wire electrode having a diameter in the range of about 50 μm to about 100 μm. In one embodiment, the electrode comprises a wire electrode having a diameter of about 75 μm. The wire electrode may have any suitable length necessary for implantation into a tissue or region of interest. In certain embodiments the electrode has a length in the range of about 1 mm-500 cm. In certain embodiments the electrode has a length in the range of about 10 mm-100 cm. In certain embodiments the electrode has a length in the range of about 1 cm-50 cm.

In certain embodiments, the electrodes comprise an outer insulation layer. In certain embodiments, the insulation layer comprises a perfluoroalkoxy Teflon (PFA) layer. Other suitable materials of the insulation layer include, but are not limited to glass, a glass coating, silicone, parylene or other suitable material known in the art. In certain embodiment, the insulation layer provides for resistance against thermal or chemical degradation of the electrode. In certain embodiment, the insulation layer provides to restriction of the sensing element(s) to specific part(s) of the wire.

In certain embodiments, the distal end of the wire electrode comprises one or more barbs, hooks, loops, or other anchoring structures to allow for anchoring of the distal tip of the wire electrode in tissue, such as the myocardium or vessel wall. For example, in one embodiment, the distal tip of the electrode is bent backwards to produce a harpoon-like structure at the electrode tip. In certain embodiments, the wire electrode in treaded through a carrier such as needle and the wire bent backwards. The needle-wire assembly can be inserted into the tissue and the carrier withdrawn, leaving the wire electrode and its sensing element embedded within the tissue. In certain embodiments, the tip of the wire electrode treaded through the carrier may have other specialized structures such as barbs on the tip to allow for anchoring of the sensor within the tissue wall when the carrier is withdrawn.

In certain embodiments, the electrode is functionalized with a receptor molecule that specifically binds to a biochemical compound of interest. The receptor molecule can be any suitable molecule, small molecule, nucleic acid, amino acid, peptide, polypeptide, antibody, antibody fragment, or the like which may recognize or selectively bind the biochemical compound or compounds of interest. The receptor molecule may be reversibly or irreversibly linked to the electrode using any suitable means known in the art. For example, the receptor molecule may be covalently or non-covalently linked to the electrode. In some embodiments, the receptor molecule is linked to the electrode using a linker molecule. In some embodiments, the linker molecule is any suitable linker molecule known in the art. In some embodiments, the linker molecule is a rigid linker. In some embodiments, the linker molecule is a flexible linker. In some embodiments, the linker is a cleavable linker. In some embodiments, the linker molecule is a polar molecule.

In certain embodiments, the device comprises one or more stimulatory electrodes to apply an electrical signal to the autonomic nervous system, sympathetic nervous system, parasympathetic nervous system, or cardiac nervous system. Exemplary electrodes include cuff electrodes, needle electrodes, and the like. In one embodiment, the system comprises one or more pacing electrodes suitable for application of cardiac electrical stimulation at one or more epicardial, endocardial or intramyocardial sites. In certain embodiments, one or more stimulating electrodes are used to induce release of a biochemical compound of interest (e.g., catecholamines) to be detected by one or more of the electrodes described herein.

In some embodiments, one or more of the electrodes is contained within a catheter. The catheter may be any suitable catheter as known in the art. In some embodiments, one or more electrodes comprise a semipermeable membrane encasing at least a portion of the electrode. In some embodiments, the semipermeable membrane creates a barrier between the electrode and the surrounding environment. In some embodiments, the semipermeable membrane comprises a porosity sufficiently large to allow biochemical compounds of interest to freely diffuse across the membrane. In some embodiments, the semipermeable membrane comprises a selectively semipermeable membrane. In some embodiments, the selectively semipermeable membrane selects for biochemical compounds of interest based on size, charge, polarity, composition, and the like. The semi-permeable membrane may be constructed from any suitable material known in the art.

In some embodiments, the device of the present invention further comprises one or more controllers, connected to supply power and signals to, and to measure signals received from, electrodes of the present invention. In one embodiment, a controller is connected to a wired communication port of an electrode, but in another embodiment the connection may be implemented via a wireless link. Power may be supplied to the controller via wires or wirelessly. In certain embodiments, the device comprises an implantable controller configured to deliver and collect signals from the one or more electrodes. The implantable controller may be in wired or wireless communication with one or more external system components. For example, in certain embodiments, the implantable controller delivers and receives information from an external computing device.

In certain embodiments, the device comprises a voltage clamp circuit operably connected to the one or more electrodes. The voltage clamp circuit may be housed in one or more controllers of the device. The voltage clamp circuit may be any voltage clamp configuration, and may be positive or negative, biased or unbiased as required by the application. As understood by one skilled in the art, a voltage clamp circuit is used to fix one or more reference potentials within pre-set limits. In one embodiment, a system of the present invention may comprise three electrodes, including a reference electrode, a ground electrode, and a sampling or measurement electrode. In some embodiments, the reference electrode and the ground electrode may be shunted together, yielding what is effectively a two-electrode configuration. In a three electrode configuration, the voltage clamp may be operably connected between the reference and ground electrodes, configured to maintain a reference voltage between the two electrodes. Separate ground and reference electrodes may be used in some embodiments to determine voltage in tissue. Such an electrode scheme may be used for example in conditions of low conductance between the sample electrode and the ground electrode—which may lead to errors in the voltage clamp and a phase offset of the obtained signals with respect to the commanded potential. Using three electrodes in such a scenario provides a more accurate voltage clamp and minimizes phase offset. This in turn leads to improved correlation between the oxidation current and the commanded potential, which provides a significantly more accurate identification of the oxidized species.

The voltage clamp may comprise a feedback resistor, and the feedback resistor may have a low resistance so as to supply adequate current to the electrodes for measurement. In one embodiment, the feedback resistor is a 1MΩ resistor. In other embodiments, the feedback resistor is a 10MΩ resistor. In some embodiments, the device is configured to have a switchable feedback resistance, where a 1MΩ or 10MΩ feedback resistor may be selected by the operator prior to scanning. In other embodiments, the feedback resistor is a potentiometer, and the feedback resistance may be selected from a continuous range of resistances. In some embodiments, the range is from 1MΩ to 10MΩ. Such low resistances may be advantageous, for example in applications where one or more electrodes are made of platinum. In such cases, the capacitance of the electrodes will be higher, and so more current will be required to charge them.

In some embodiments, a device of the present invention comprises multiple sampling or measurement “channels” from which data is gathered simultaneously or in alternating sequence. The multiple channels may share a single reference electrode and ground electrode, or may alternatively be split among multiple reference and/or ground electrodes. Each channel has at least one distinct measurement electrode, and the various measurement electrodes may be positioned in different areas of the tissue being measured in order to simultaneously monitor relevant concentrations across a larger area. Measurement electrodes may be substantially similar to the reference and ground electrodes, or may alternatively have a different size, shape, cross-sectional area, or material than the reference and ground electrodes. In some embodiments, the ground, reference, and measurement electrodes are all made from different materials or in different shapes. In some embodiments, the reference and ground electrodes are made from steel. In some embodiments, the reference electrodes are made from silver or silver chloride. In some embodiments, one or more of the electrodes are made from platinum.

In certain embodiments, the device comprises one or more potentiostats operably connected to the one or more electrodes. In certain embodiments, the one or more potentiostats are housed in one or more controllers of the device. As described herein, a potentiostat is a circuit configured to impose a voltage across two or more electrodes while measuring the current passing through a lead connected to one or more of the electrodes. A command potential (scanning voltage waveform) is used to control the voltage on the measurement electrode with respect to the tissue voltage measured from the ground and/or reference electrodes. The command potential may be asserted by any method known in the art, including but not limited to a function generator, timing circuit, or via a digital-to-analog converter (DAC). In one embodiment, a USB controlled multi-channel DAC is used. DACs provide fast switching and voltage control, but may suffer in some cases from quantization errors. That is, analog curved waveforms, for example sine waves, will look imperfect when examined at high magnification because DACs are capable only of generating a finite set of voltage values. This is particularly true if a low-resolution DAC, for example an 8-bit DAC, is used, but the effect is still present in other DACs appropriate for use in the present invention, including but not limited to a 10-bit DAC, a 12-bit DAC, a 16-bit DAC, or a 24- or 32-bit DAC. In some embodiments, the effect of the quantization error may be mitigated by inducing a higher peak-to-peak voltage from the DAC than is required, then scaling the higher voltage down using, for example, a voltage divider and follower as known in the art. Suitable scaling factors will vary based on the capabilities of the DAC used and the voltage range required by the application, but exemplary scaling factors may be 2×, 5×, 10×, 20×, or 50×. The scaling factor in any particular device of the present invention may be fixed, or may alternatively be switchable among multiple values to allow for greater fidelity and dynamic range in command potential. In some embodiments, the voltage clamping function described above is performed by the one or more potentiostats. Alternatively, a single circuit or set of integrated circuits and passive components may perform both the functions of the potentiostat and the functions of the voltage clamp as described herein.

Embodiments of the invention using DACs are advantageous because they may be easily synchronized with a corresponding analog-to-digital converter (ADC) used for data acquisition. In some embodiments, a single computer-controlled data acquisition device may be used, including one or more DACs to generate the command potential and one or more ADCs for reading data back from the device. In one embodiment, the ADCs are connected across a sensing resistor having a precise, known resistance, and record the current resulting from the oxidation or reduction of the various compounds as a voltage level across the sense resistor.

Exemplary command potentials for use with the present invention include but are not limited to sine waves, sawtooth waves, and square waves. The frequency of the command potential may in some embodiments be between 1 Hz and 50 Hz, or between 2 Hz and 25 Hz, or between 5 Hz and 20 Hz. Suitable amplitudes include 1.7 volts peak to peak (Vpp), 1 Vpp, 0.5 Vpp, 2 Vpp, or any other voltage adequate to capture concentration-dependent currents at characteristic oxidation potentials.

One exemplary embodiment of the invention is directed to the measurement of the concentration of norepinephrine, which has an oxidation voltage of approximately 400 mV, releasing two electrons per molecule when it oxidizes. In this embodiment, the command potential has a Vpp of 1.7V, and a positive bias of 350 mV, resulting in a maximum voltage of +1.2V and a minimum voltage of −500 mV.

Systems of the present invention may further comprise one or more signal processing modules including but not limited to filtering, amplification, storage, and analysis modules, connected via wires or wirelessly to one or more electrodes. In some embodiments, the various signal processing modules are implemented as dedicated hardware circuitry, but the signal processing functions may also be implemented as software on a computing device. The purpose of the signal processing modules is to generate data and draw inferences from the measurements gathered from the various probes of the present invention. Filtering modules may include, but are not limited to high-pass, low-pass, or band-pass filters, Kalman filters, or any other filtering module used in the art. Amplification modules of the present invention may comprise one or more operational amplifiers or transistors, or may alternatively accomplish amplification through software means such as multiplication of analog values to add gain to some or all of the signals received. Storage modules may include any suitable means of data storage, including but not limited to hard disk drives, solid state storage, or flash memory modules.

The various sensors described herein may return measurements to a collection device as analog voltage levels, digital signals, or both. As described herein, “collection device” refers to any device capable of receiving analog or digital signals and performing at least one of: storing the data on a non-transitory computer-readable medium or, transmitting the data via a wired or wireless communication link to a remote computing device. In some embodiments, the collection device may further comprise a processor and stored instructions for performing analysis or display of the data collected. In some embodiments, the system further comprises a graphical user interface (GUI) and a display capable of presenting some or all of the data, or calculated derivatives thereof, in human readable form. The data collected may be presented as a time series kymograph, real-time display of current values, minimum or maximum values, or any other display format known in the art.

Exemplary GUIs of the present invention may include one or more controls, including Boolean, numerical, sliding, or rotary controls, for manipulation of various parameters related to systems and methods of the present invention. Examples of parameters that may be controlled by computer-implemented GUIs of the present invention include dynamic amplifier or potentiostat parameters, parameters of the command potential (including but not limited to the start potential, end potential, frequency, rate of scan, amplitude, and step size), and data measurement or acquisition parameters including but not limited to sampling granularity, sampling frequency, significant digits, and recording mode. In some embodiments, a GUI of the present invention may present a set of measurements as a time-series kymograph. In other embodiments, data may be presented as a list of numerical values, or a frequency-domain graph.

Software applications of the present invention may also include one or more analysis modules, configured to perform signal or data processing steps on the raw data collected by the measurement or acquisition modules of the present invention. In one example, an analysis module may isolate oxidation- or reduction-specific signals from the capacitive currents inherent in the electrode. In another embodiment, an analysis module may perform noise detection and correction steps to remove unwanted noise from the recorded signal. In another embodiment, an analysis module may perform a frequency domain analysis of a collected time series signal, or may detect the relative position of peaks in a set of measured time-domain voltage or current values, using the position and magnitude of the located peaks to automatically determine the concentration of one or more compounds near the measurement electrode over time.

### Methods

The present invention as described herein provides methods for detecting, measuring, or monitoring the presence and abundance of one or more biochemical compounds. For example, as described herein, the present invention enables detection of one or more compounds of interest with high spatial and temporal resolution.

The method comprises the detection of any suitable biochemical compounds of interest, including, but not limited to neurotransmitters, proteins, peptides, nucleic acid molecules, hormones, lipids, ions, and the like.

In some embodiments, the method is used for the detection of specific proteins in the heart, including but not limited to Enkephalins, Neuropeptide Y, substance P, calcitonin gene-related peptide (CGRP), and brain natriuretic peptide (BNP). In certain embodiments, the method is used for the detection of neurotransmitters, including, but not limited to catecholamines, such as norepinephrine, epinephrine, and acetylcholine.

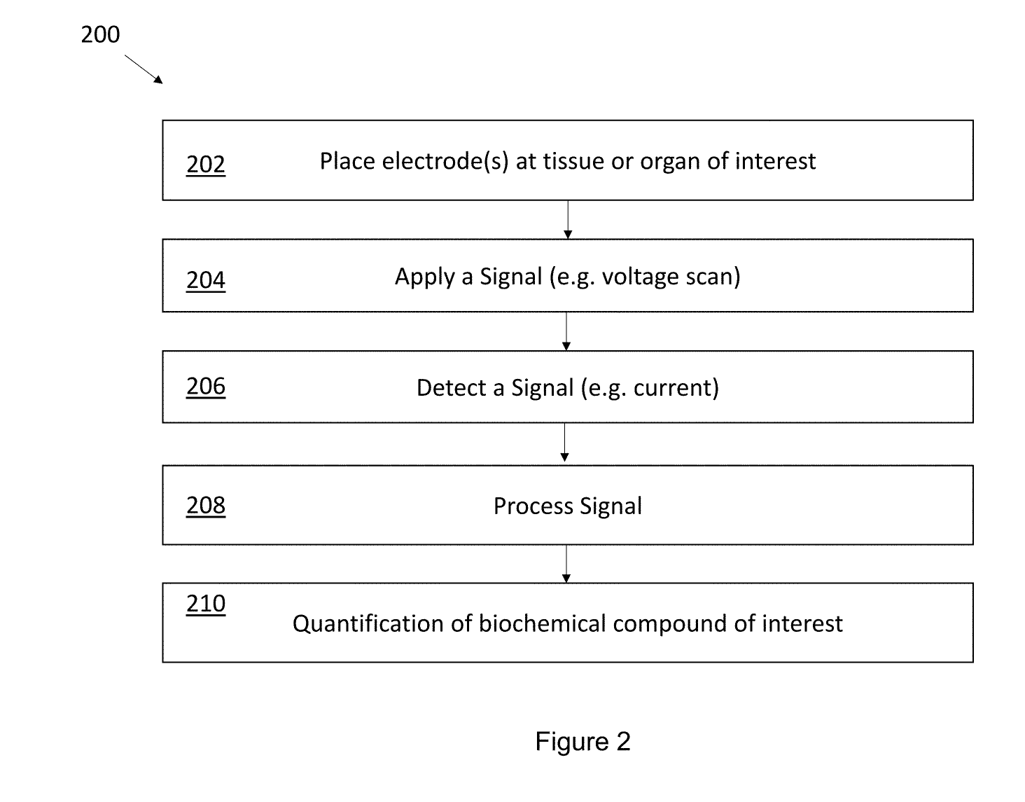

Referring now to FIG. 2, an example process 200 for detecting the presence and abundance of a biochemical compound of interest is shown. One or more steps of process 200 may be implemented, in some embodiments, by one or more components of the system and device, as described herein. In some embodiments, as depicted in block 201, the method comprises placing one or more electrodes, as described herein, within a region of interest. The one or more electrodes may be placed in any suitable location to detect the biochemical compounds of interest.

In some embodiments, the region of interest is one or more locations within the myocardium. In some embodiments, the region of interest is adjacent to an organ or tissue of interest. In some embodiments, the region of interest is adjacent to one or more nerves, nerve divisions, ganglia or regions of a nerve of interest. In some embodiments, the region of interest is within one or more nerves, ganglia, nerve divisions and the like. In some embodiments, the one or more electrodes are placed into vascular space in proximity to the organ or tissue of interest. In some embodiments, the one or more electrodes is placed into interstitial space in proximity to an organ or tissue of interest. In some embodiments, the one or more electrodes are placed into a chamber of the heart, for instance the right atrium, the right ventricle, the left atrium, and/or the left ventricle. In some embodiments, the one or more electrodes are placed into a blood vessel, for example, inferior vena cava, superior vena cava, coronary sinus, coronary artery, coronary vein, ascending aorta, aorta, pulmonary artery, pulmonary vein, great veins of the heart, a peripheral vein, a peripheral artery and the like. In some embodiments, the one or more electrodes are placed into the pericardial space.

For example, in certain embodiments, one or more electrodes are placed in the atrial myocardium, ventricular myocardium, vascular space of the heart, coronary sinus of the heart, left ventricle, right ventricle, left atrium, right atrium, epicardial fat pad, pericardial fat pad, aorta, pulmonary vein, pulmonary artery, vena cava, or the like. In certain embodiments, one or more electrodes can be placed within a neural structure, including at a neural structure of the autonomic nervous system, such as at one or more of a peripheral nerve, the intrathoracic ganglia, stellate ganglia, autonomic ganglia, nodose ganglia, dorsal root ganglia, petrosal ganglia, or sensory ganglia. In various embodiments, the method comprises placement of one or more electrodes at different locations within the autonomic nervous system and/or heart to detect regional differences

in the abundance of one or more biochemical compounds of interest.

In one embodiment, the method comprises inserting one or more wire electrodes into a region of interest. For example, in one embodiment, the method comprises inserting a wire electrode through the distal tip of a needle, inserting the needle through cardiac tissue, and withdrawing the needle, thereby leaving the electrode within the tissue. In some embodiments, prior to insertion of the needle, the wire is advanced past the needle tip, and the wire is bent backwards along the shaft of the needle forming a harpoon-like shape, enabling the electrode to remain in the tissue while the needle is withdrawn. In some embodiments, the distal tip of the electrode comprises one or more anchoring structures, as described elsewhere herein, thereby allowing the electrode to remain in the tissue while the needle is withdrawn.

In some embodiments, as depicted in block 204, the method of the invention further comprises applying a signal to one or more electrodes. In certain embodiments, the method comprises the use of voltammetry, including, but not limited to fast scanning cyclic voltammetry (FSCV), potential step voltammetry, linear sweep voltammetry, cyclic voltammetry, square wave voltammetry, staircase voltammetry, anodic or cathodic stripping voltammetry, adsorptive stripping voltammetry, alternating current voltammetry, rotated electrode voltammetry, normal or differential pulse voltammetry, chronoamperometry, and chronocoulometry. In some embodiments, an FSCV signal is applied to one or more electrodes.

In certain embodiments, a control unit or controller is configured to deliver a signal to one or more electrodes. The signal may comprise a constant voltage or a specific pattern of variable voltage. For example, in certain embodiments, the method comprises delivering a pattern of increasing and decreasing voltages (i.e., voltage scanning) in a step, triangular, sinusoidal, saw tooth, or any other suitable pattern. In FSCV applications, the method comprises rapidly increasing and decreasing the voltage at the electrode tip. In certain embodiments, the method comprises administering a cyclic voltage signal, where the applied pattern of voltage is repeated for a defined duration or number of periods. In some embodiments, the signal is applied at a frequency of less than 1 Hz, 1 Hz to 50 Hz, or greater than 50 Hz. In one embodiment, the signal is applied at a frequency in the range of about 1 Hz to 50 Hz.

In certain embodiments, the delivered voltage scans between a minimum voltage of about −5V to −200 mV and a maximum voltage of about 200 mV to 5V. In one embodiment, the delivered voltage scans between about −500 mV to about 1.2V. In one embodiment, the voltage scans can be delivered at rate of about 1-50 V/s. In one embodiment, the voltage scans can be delivered at rate of about 5-20 V/s.

In some embodiments, as depicted in block 206, the method comprises detecting a signal from one or more electrodes. For example, in certain embodiments, the method comprises detecting a current in response to the delivered voltage signal. In certain embodiments, the method comprises measuring a current using the same electrode that was used to deliver the voltage. In certain embodiments, the method comprises detection of current indicative of the oxidation and/or reduction of the biochemical compound of interest. As described elsewhere herein, the delivered voltage scan results in the oxidation and reduction of biochemical compounds in the vicinity of the electrode tip which produces a current overlaid on the background current detected by the electrode.

In certain embodiments, where the electrode is functionalized with a receptor molecule, the presence of a biochemical compound of interest that specifically binds to the receptor molecule is observed by detecting a change in the capacitance of the electrode. For example, in certain aspects, binding of the compound of interest to the receptor molecule increases or decreases the native capacitance of the electrode. The change in capacitance can be measured in any suitable manner. For example, in certain embodiments, the capacitance of the electrode can be measured by delivering voltage steps to the electrode and measuring the time constant of the electrode, thereby enabling the calculation of the capacitance, a parameter that changes upon detection and binding of the molecule of interest. In one embodiment, the capacitance of the electrode can be measured by measuring a current or a change in a current. In other embodiments, capacitance of single equivalent circuits are measured in a frequency-domain analysis allowing for spectral unmixing of multiple signals on a single electrode, each specific for a single molecule of interest. Such an embodiment would be designed by attaching more than one trap molecule (eg. antibody) to the tip of the electrode, thus allowing for the measure of multiple molecules of interest simultaneously.

In some embodiments, as depicted in block 208, the method comprises processing one or more signals detected from the one or more electrodes. In certain embodiments, a control unit or controller may process the signal so that the detected signal is recorded or displayed as a voltage, current, capacitance, or any other relevant parameter.

In certain embodiments, as depicted in block 210, the method comprises processing the signal to produce a voltammogram of detected current as a function of voltage. In one embodiment, a voltammogram is produced by subtracting baseline current from the detected current, in response to an applied voltage scan, thereby producing the oxidation current induced by the biochemical compound of interest. In certain embodiments, one or more characteristics of the voltammogram are used to identify the compound. For example, as shown in FIG. 6A and FIG. 6B, the oxidation of norepinephrine produces a single peak, while the oxidation of epinephrine produces two peaks. Therefore, in certain embodiments, the method comprises comparing the voltammogram with a standard or reference voltammogram to identify the one or more detected compounds.

In certain embodiments, the method comprises quantifying the amount of the biochemical compound of interest. For example, in certain embodiments, the method comprises identifying the peak current, where the amplitude of the peak current can be used to calculate the concentration of the compound of interest. For example, in certain embodiments, a standard curve or calibration curve is used to calculate the concentration of the compound of interest. The standard curve or calibration curve can be based upon the peak amplitudes detected in the in vitro or ex vivo detection of known concentrations of the compound of interest. Use of a standard curve to calculate the concentration of detected norepinephrine and epinephrine is shown in FIG. 7.

In some embodiments, the method comprises recording and storing the detected signal. In certain embodiments, the method comprises recording and storing the detected signal and the applied signal (e.g., voltage scan).

In some embodiments, the detected signal may be processed in order to determine trends in the detected signal. For example, the detected signal may be processed as voltage with respect to time, as voltage with respect to current, as current with respect to time, and the like, as known in the art. In some embodiments, calibration curves may be computed from the detected signal. For example, the signal (i.e. current, voltage, capacitance, etc.) that is detected when the sensor is placed in proximity to known concentrations of a biological compound of interest may be used in order to calibrate the detected signal to one or more known concentrations. In some embodiments, the computed calibration curves may be used in order to quantify the concentration of an unknown amount of a biological compound of interest. In some embodiments, the controller automatically generates calibration curves that may be used to compute concentrations of unknown amounts of biological compounds. In some embodiments, the calibrated concentration of a detected biological compound may be displayed on the user interface of the controller. In some embodiments, the sensor may be calibrated in order to determine whether a biological compound is detected or not. In some embodiments, the detected signal and/or processed signal may be stored by the controller. In some embodiments, the detected signal and/or processed signal may be transferred by means known in the art to an external device.

In certain embodiments, the present invention provides a method of detecting or monitoring the level of a biochemical compound of interest, such as a neurotransmitter or protein or peptide of interest, in response to one or more cardiac stressors or other stimulation. In one embodiment, the one or more cardiac stressors comprises transient reductions or increases in cardiac preload (venous return). In one embodiment, the one or more cardiac stressors comprise a transient increase or decrease in cardiac afterload (arterial blood pressure). In one embodiment, the one or more cardiac stressors comprise increases or decreases in sympathetic efferent inputs to the heart. For example, in certain embodiments, a change in sympathetic efferent inputs to the heart is achieved by stimulation or local block of intrathoracic sympathetic projections to the heart. In certain embodiments, a change in sympathetic efferent inputs to the heart is achieved by stimulation or block of the dorsal aspect of the spinal cord. In one embodiment, the one or more cardiac stressors comprise increases or decreases in parasympathetic efferent inputs to the heart. In certain embodiments, a change in parasympathetic efferent inputs is achieved by stimulation or local block of parasympathetic efferent projections to the heart. In one embodiment, the one or more cardiac stressors comprises increases or decreases in autonomic control of the heart. For example, in one embodiment a change in the autonomic control of the heart is achieved by stimulation or local block of intrinsic cardiac ganglia. In one embodiment, the one or more cardiac stressors comprise increases or decreases in cardiac afferent input. For example, in one embodiment a change in the cardiac afferent input is achieved by stimulation or local block of intrathoracic sensory input to autonomic ganglia. In one embodiment, a change in afferent input is achieved by stimulation or block of nodose afferent neurons. In one embodiment, a change in afferent input is achieved by stimulation or block of dorsal root ganglia. In one embodiment, the more or more cardiac stressors comprises cardiac pacing. Such cardiac pacing may be from electrodes placed on or in the atrium, ventricles or both. In one embodiment, the pacing may be condition-test pacing where a set of conditioned pace beats is followed by one or more pace stimuli of shorter inter-pace interval. In one embodiment, the pacing may be decremental with progressive decreases in inter-pace intervals. In one embodiment, the pacing may be burst type pacing with burst frequencies between 1 to 10 Hz. In one embodiment, the pacing may be synchronized to cardiac electrical activity to deliver a single or multiple pulses at cycle lengths less than the basal heart rate cycle length; such pacing stimuli modeling premature atrial and ventricular electrical events. In one embodiment, chemicals that modulate cardiomyocyte or neural activity may be placed on the heart or injected into the vascular space. In one embodiment, changes in ventilation may be used as a transient cardiopulmonary stress. In one embodiment, changes in ventilation may include one or more of the following, changes in ventilation rate, ventilation tidal volume, outflow pressure and inflow gas mixture.

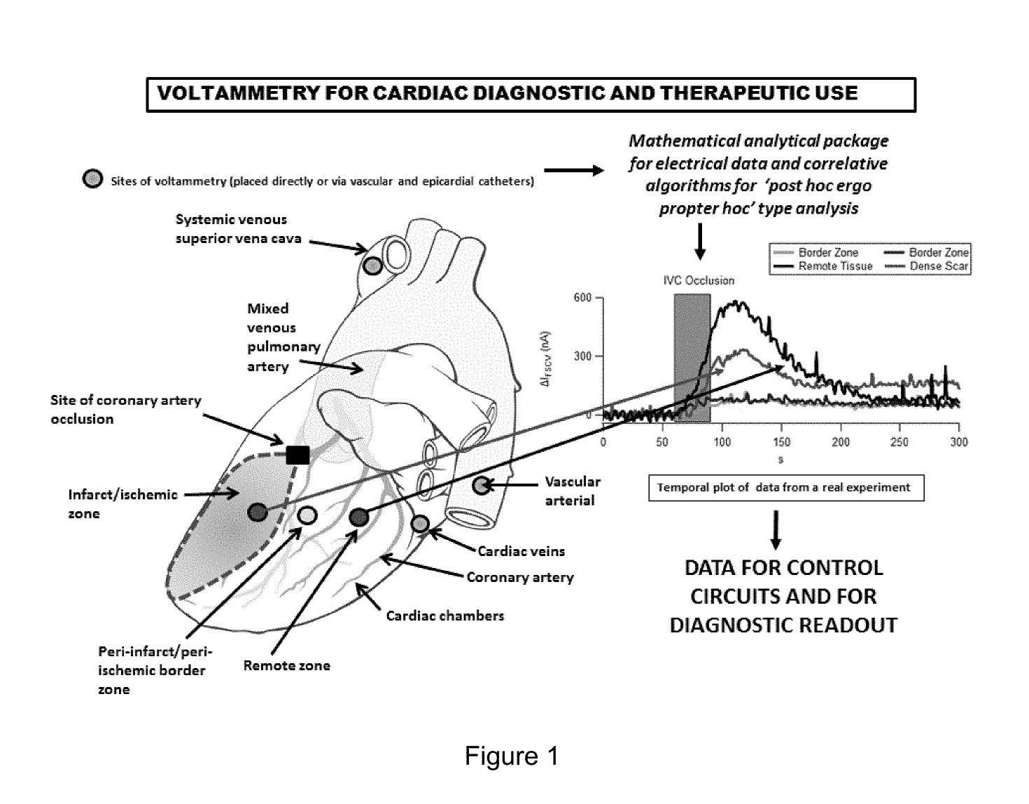

In one aspect, the invention relates to a method for monitoring cardiac or cardiopulmonary autonomic function or dysfunction, comprising inserting one or more electrodes into a myocardium and applying a voltage scan (e.g. a FSCV signal) to measure neurotransmitter (e.g., catecholamine) levels in the vicinity of the tip of the electrode. In certain embodiments, the one or more electrodes are placed into the atrial myocardium or into the ventricular myocardium. The electrode or electrodes may be placed from vascular access or epicardial access. FIG. 1 illustrates an exemplary distribution of interstitial recording electrodes placed into the ventricles. However, the present invention is not limited to the particular distribution depicted in FIG. 1.

In another aspect, the invention relates to a method for monitoring cardiac or cardiopulmonary autonomic function or dysfunction, comprising inserting a catheter-based electrode into vascular space of a heart, and applying a voltage scan (e.g., a FSCV signal) to measure neurotransmitter (e.g., catecholamine) content in the vicinity of the catheter-based electrode. In certain instances the catheter-based electrode is an FSCV sensor. In one embodiment, the catheter-based electrode is placed in a coronary sinus of the heart to measure neurotransmitter levels at the immediate venous outflow from the heart. In one embodiment, the catheter-based electrode is placed in the great veins of the heart to measure neurotransmitter (e.g. catecholamine) levels at the inflow to the heart. In one embodiment, the catheter-based electrode is placed in the left ventricle of the heart or the aorta to measure neurotransmitter (e.g., catecholamine) levels before entry to the coronary vasculature of the heart. In one embodiment, the catheter-based electrode is placed in the right ventricle of the heart or a pulmonary artery to measure neurotransmitter (e.g., catecholamine) levels before entry to the pulmonary vasculature of the heart. In one embodiment, the catheter-based electrode is placed in the left atrium or pulmonary veins to measure neurotransmitter (e.g. catecholamine) levels after exit from the pulmonary circulation. In one embodiment, a plurality of catheter-based electrodes are placed in one or more of a coronary sinus, cardiac chambers, vena cava or aorta of the heart to measure trans-cardiac neurotransmitter (e.g., catecholamine) levels. In one embodiment, a plurality of catheter based electrodes are placed into one for more of the right atria, right ventricle or pulmonary artery (e.g. inflow to pulmonary circuit) and pulmonary veins or left atria (e.g. outflow from pulmonary circuit) to measure trans-pulmonary neurotransmitter (e.g. catecholamine) levels. In one embodiment, the catheter-based electrode is placed directly in blood. In one embodiment, the method comprises inserting a catheter-based electrode into vascular space and applying a voltage scan (e.g., FSCV signal) to measure neurotransmitter (e.g. catecholamine) content in the vicinity of the recording sensor in response to one or more cardiac stressors or stimulation, as described above. In one embodiment, the local, transcardiac and transpulmonary basal neurotransmitter (e.g. catecholamine) levels are assessed in the vascular compartment. In one embodiment, the local, transcardiac and transpulmonary neurotransmitter (e.g. catecholamine) levels are assessed in the vascular compartment in response to one or more cardiac stressors or stimulation, as described above.

In one embodiment, a semi-permeable membrane is placed between the catheter-based electrode and blood. For example, in certain embodiments, the catheter-based electrode comprises a semi-permeable membrane. In one embodiment, the pore size of the semi-permeable membrane is sufficient to allow passage of neurotransmitter (e.g., catecholamine) from the blood to the vicinity of the electrode. FIG. 1 illustrates a representative distribution of the vascular recording sensors placed in vessels adjacent to and within the heart.

In another aspect, the present invention relates to a method of assessing regional differences in autonomic control of regional cardiac function or dysfunction. In one embodiment, the method comprises inserting multiple electrodes into a myocardium of a heart and applying a voltage scan (e.g., an FSCV signal) to measure regional levels in a local vicinity of a tip of the electrode. In one embodiment, regional basal neurotransmitter (e.g., catecholamine) levels are assessed. In one embodiment, regional neurotransmitter (e.g., catecholamine) levels are assessed in response to one or more cardiac stressors or stimulation, as described above. FIG. 1 depicts a representative catecholamine release profile into the ventricular interstitium in response to a decrease in preload produced by transient occlusion of the inferior vena cava.

In another aspect, the present invention provides a method for measuring neurotransmitter (e.g., catecholamine) levels in the peripheral blood, comprising inserting an electrode into a blood vessel and applying a voltage scan (e.g., a FSCV signal) to measure neurotransmitter (e.g., catecholamine) levels in the vicinity of a tip of the electrode. In one embodiment, the electrode is placed into a peripheral artery. In one embodiment, the electrode is placed into a peripheral vein. In one embodiment, the electrode is a catheter-based electrode. In one embodiment, the electrode is placed from vascular access. In one embodiment, a semi-permeable membrane is placed between the catheter-based electrode and blood. For example, in certain embodiments, the catheter-based electrode comprises a semi-permeable membrane. In one embodiment, the pore size of the semi-permeable membrane is sufficient to allow passage of neurotransmitter (e.g., catecholamine) from the blood to the vicinity of the electrode.

In one aspect, the present invention provides a method for monitoring cardiac or cardiopulmonary autonomic function or dysfunction, comprising inserting one or more functionalized electrodes (e.g., capacitive immunosensors) into a myocardium and applying a signal (e.g., voltage) to the functionalized electrode to measure the level of a protein or peptide of interest in the local vicinity of the tip of the functionalized electrode. In certain embodiments, the one or more functionalized electrodes are placed into the atrial myocardium, into the ventricular myocardium or both. The functionalized electrode or electrodes may be placed from vascular access or epicardial access.

In another aspect, the invention relates to a method for monitoring cardiac or cardiopulmonary autonomic function or dysfunction, comprising inserting a catheter-based functionalized electrode into vascular space of a heart, and applying a signal (e.g., voltage) to measure the level of a protein or peptide of interest in the vicinity of the catheter-based functionalized electrode. In one embodiment, the catheter-based functionalized electrode is placed in a coronary sinus of the heart to measure the level of a protein or peptide of interest at the immediate venous outflow from the heart. In one embodiment, the catheter-based functionalized electrode is placed in the great veins of the heart to measure the level of a protein or peptide of interest at the inflow to the heart. In one embodiment, the catheter-based functionalized electrode is placed in the left entricle of the heart or the aorta to measure the level of a protein or peptide of interest before entry to the coronary vasculature of the heart. In one embodiment, the catheter-based functionalized electrode is placed in the right ventricle of the heart or a pulmonary artery to measure the level of a protein or peptide of interest before entry to the pulmonary vasculature of the heart. In one embodiment, the catheter-based functionalized electrode is placed in the left atrium or pulmonary veins to measure the level of a protein or peptide of interest after exit from pulmonary vascular circuit. In one embodiment, a plurality of catheter-based functionalized electrodes are placed in one or more of a coronary sinus, cardiac chambers, vena cava or aorta of the heart to measure the trans-cardiac level of a protein or peptide of interest. In one embodiment, a plurality of catheter-based functionalized electrodes are placed in one of more of a great vein, right atria, right ventricle, pulmonary artery, pulmonary vein, left atria or left ventricle to measure the trans-pulmonary level of a protein for peptide of interest. In one embodiment, the catheter-based functionalized electrode is placed directly in blood. In one embodiment, the method comprises inserting a catheter-based functionalized electrode into vascular space and applying a signal (e.g., voltage) to the level of a protein or peptide of interest in the vicinity of the recording sensor in response to one or more cardiac stressors or stimulation, as described above. In one embodiment, the local, transcardiac or transpulmonary basal level of a protein or peptide of interest are assessed in the vascular compartment. In one embodiment, the local, transcardiac and/or transpulmonary levels of a protein or peptide of interest are assessed in the vascular compartment in response to one or more cardiac or pulmonary stressors or stimulation, as described above.

In one embodiment, a semi-permeable membrane is placed between the catheter-based functionalized electrode and blood. For example, in certain embodiments, the catheter-based functionalized electrode comprises a semi-permeable membrane. In one embodiment, the pore size of the semi-permeable membrane is sufficient to allow passage of a protein or peptide of interest from the blood to the vicinity of the functionalized electrode.

In one embodiment, the present invention provides a method of assessing a regional difference in autonomic control of regional cardiac function. In one embodiment, the method comprises inserting a plurality of functionalized electrodes into the myocardium, autonomic ganglia, or sensory ganglia. In one embodiment, the method comprises applying functionalized electrodes to measure the regional levels of one or more proteins or peptides of interest in the local vicinity of the tip of each functionalized electrode. In one embodiment, regional cardiac interstitial basal protein or peptide transmitter levels are assessed. In one embodiment, regional cardiac interstitial protein or peptide transmitter levels are assessed in response to cardiac stressors, pulmonary stressors or stimulation as described above. In one embodiment, interstitial protein or peptide levels are assessed in one or more of intrathoracic autonomic, stellate, nodose, dorsal root, and/or petrosal ganglia at baseline and in response to cardiac stressors, pulmonary stressors or stimulation as described above.

In another aspect, the present invention provides a method for measuring the level of a protein or peptide of interest in the peripheral blood, comprising inserting one or more functionalized electrodes into a blood vessel and applying a signal (e.g., voltage) to measure the levels of one or more proteins or peptides of interest in the vicinity of the tip of each functionalized electrode. In one embodiment, the electrode is placed into a peripheral artery. In one embodiment, the electrode is placed into a peripheral vein. In one embodiment, the functionalized electrode is a catheter-based functionalized electrode. In one embodiment, the functionalized electrode is placed from vascular access. In one embodiment, a semi-permeable membrane is placed between the catheter-based functionalized electrode and blood. For example, in certain embodiments, the catheter-based functionalized electrode comprises a semi-permeable membrane. In one embodiment, the pore size of the semi-permeable membrane is sufficient to allow passage of a protein or peptide of interest from the blood to the vicinity of the functionalized electrode.

In certain embodiments, the present invention provides a method for detection of a cardiac defect or cardiac dysfunction in a subject by measuring one or more biochemical compounds. For example, in certain embodiments, the method comprises detecting a cardiac defect or cardiac dysfunction using one or more of the electrodes described herein to detect a neurotransmitter (e.g., catecholamines) or protein or peptide of interest. For example, as described herein, LAD occlusion resulted in the observation of increased concentrations of norepinephrine measured using voltammetry. Thus, the methods of the present invention can be used to detect cardiac dysfunction including, but not limited to, myocardial infarction, great vessel occlusion and modulation of autonomic inputs to the heart In certain embodiments, the ability to measure regional differences in catecholamines in addition to other neuromodulators and hormones provides greater insights into normal and abnormal function of the neural-heart interface that can be predictive of adverse outcomes, including potential for arrhythmias and heart failure. In certain embodiments, the ability to measure regional differences in catecholamines in addition to other neuromodulators and hormones provides a methodology to rapidly assess efficacy to therapeutic interventions. In certain embodiments, the ability to measure regional differences in the vascular compartment for catecholamines in addition to other neuromodulators and hormones provides greater insight into relevant biomarkers indicative of susceptibility to cardiac pathology and the progression of the cardiovascular disease process.

In one embodiment, the present invention provides a method for treating or preventing a cardiac defect or dysfunction in a subject, based upon the detection of one or more biochemical compounds. In certain embodiments, the method comprises treating the subject with at least one therapeutic element upon the detection of an aberrant level or pattern of one or more biochemical compounds. In certain embodiments, the treatment may include the administration of a drug, compound or other chemical or biological material. In certain embodiments, the treatment may include administration of an electrical stimulus or other forms of energy including, but not limited to, focal temperature changes, radiofrequency, electromagnetic radiation, infrared radation, or ultrasound, to one or more regions of the heart, including any myocardial tissues or any intrinsic neurons associated therewith. In certain embodiments, the treatment may be administered to extracardiac nexus points including, but not limited to the intrathoracic ganglia, the vagosympathetic trunk, and the spinal cord.

### EXPERIMENTAL EXAMPLES

The invention is further described in detail by reference to the following experimental examples. These examples are provided for purposes of illustration only, and are not intended to be limiting unless otherwise specified. Thus, the invention should in no way be construed as being limited to the following examples, but rather, should be construed to encompass any and all variations which become evident as a result of the teaching provided herein.

Without further description, it is believed that one of ordinary skill in the art can, using the preceding description and the following illustrative examples, make and utilize the present invention and practice the claimed methods. The following working examples therefore, specifically point out exemplary embodiments of the present invention, and are not to be construed as limiting in any way the remainder of the disclosure.

### Example 1

### Real Time Catecholamine Detection in the Heart

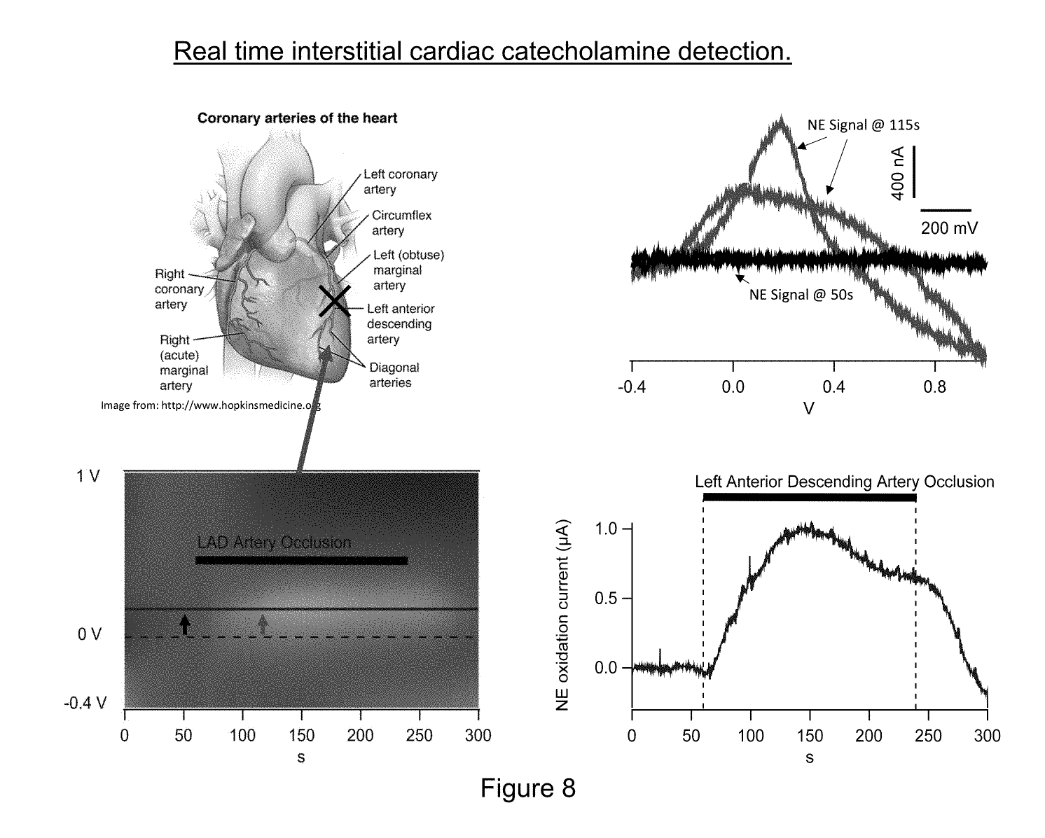

Experiments were conducted to examine whether catecholamines can be detected within the heart using FSCV. A flexible electrode was implanted into the ventricular wall of the beating heart of an anesthetized pig. The left anterior descending (LAD) artery was occluded above the implanted electrode, and norepinephrine was measured by the electrode using FSCV. A kymograph (FIG. 8) was created depicting oxidation potential plotted over voltage and time. In response to LAD occlusion, an increase in current is observed at the primary oxidation potential that lasts the duration of the occlusion before dissipation. Analysis of voltammograms at defined time points, before and during LAD occlusion, allows for visualization of peak potentials of the oxidation potential. Plotting the primary oxidation potentional for norepinephrine as a function of time demonstrates the real-time dynamics of norepinephrine detection during LAD occlusion (FIG. 8).

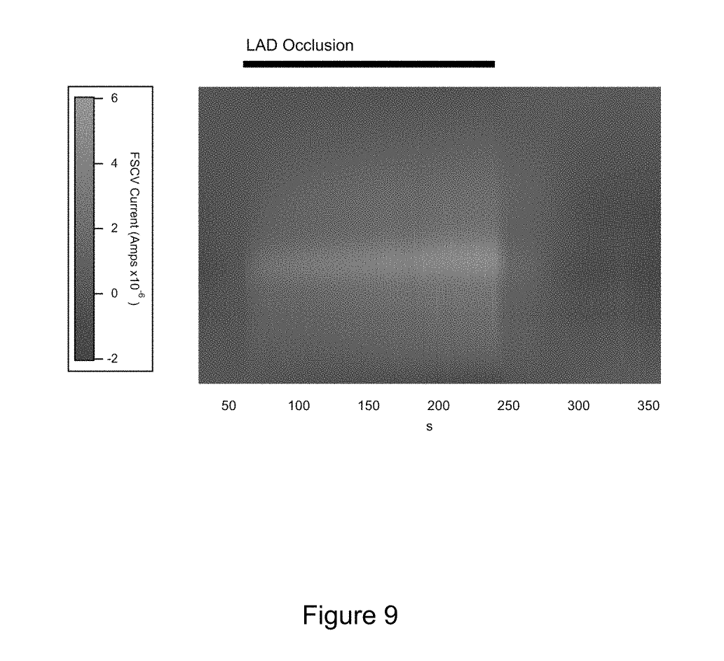

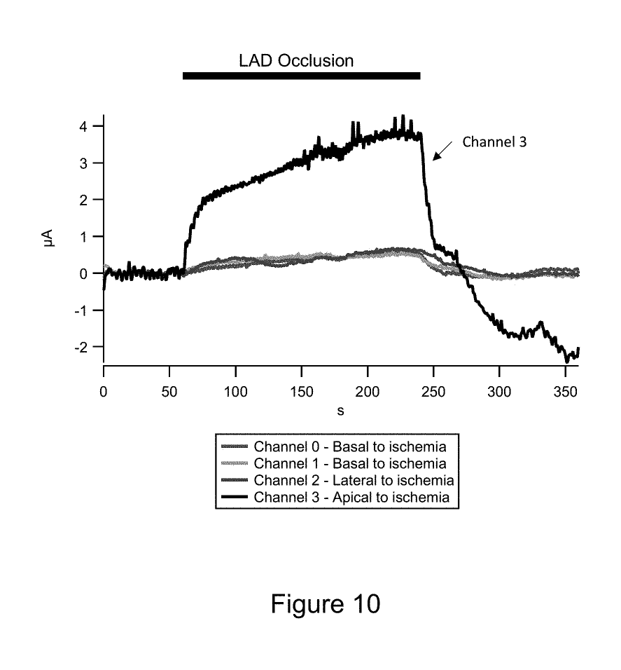

Experiments were also conducted using multiple electrodes positioned in different regions of the heart to measure norepinephrine in the heart during LAD occlusion. FSCV currents were measured in regions of the heart relative to the induced ischemic zone. FIG. 9 depicts a kymograph from one of the electrodes prior to, during, and following manual arterial occlusion protocol, demonstrating an increased oxidation current characteristic for norepinephrine. FIG. 10 depicts the data from all 4 channels, demonstrating the ability to measure FSCV at high time resolution in sub regions of the heart.

### Example 2

### Peptide Detection

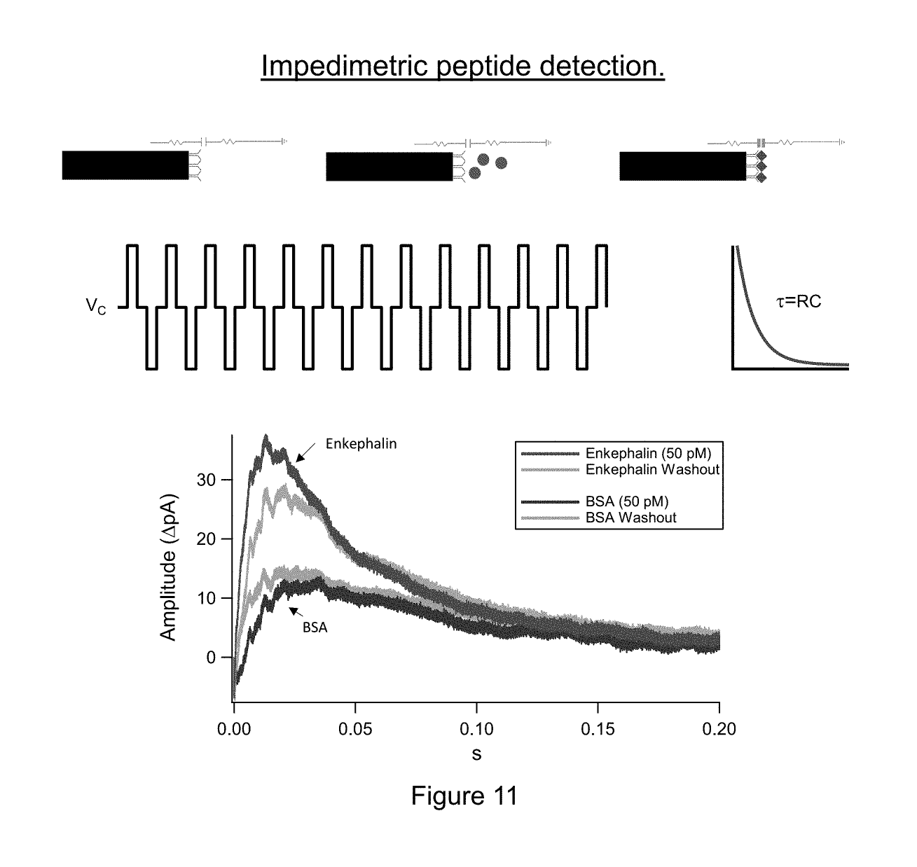

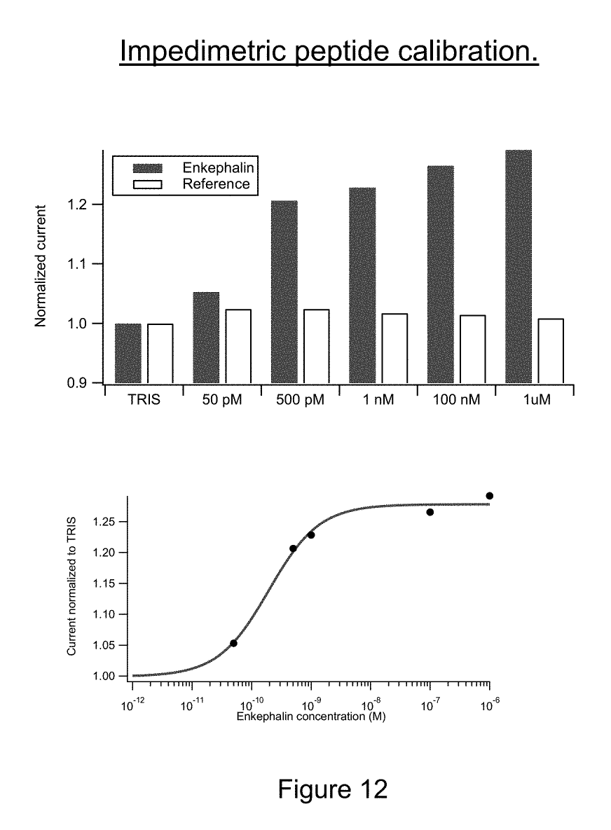

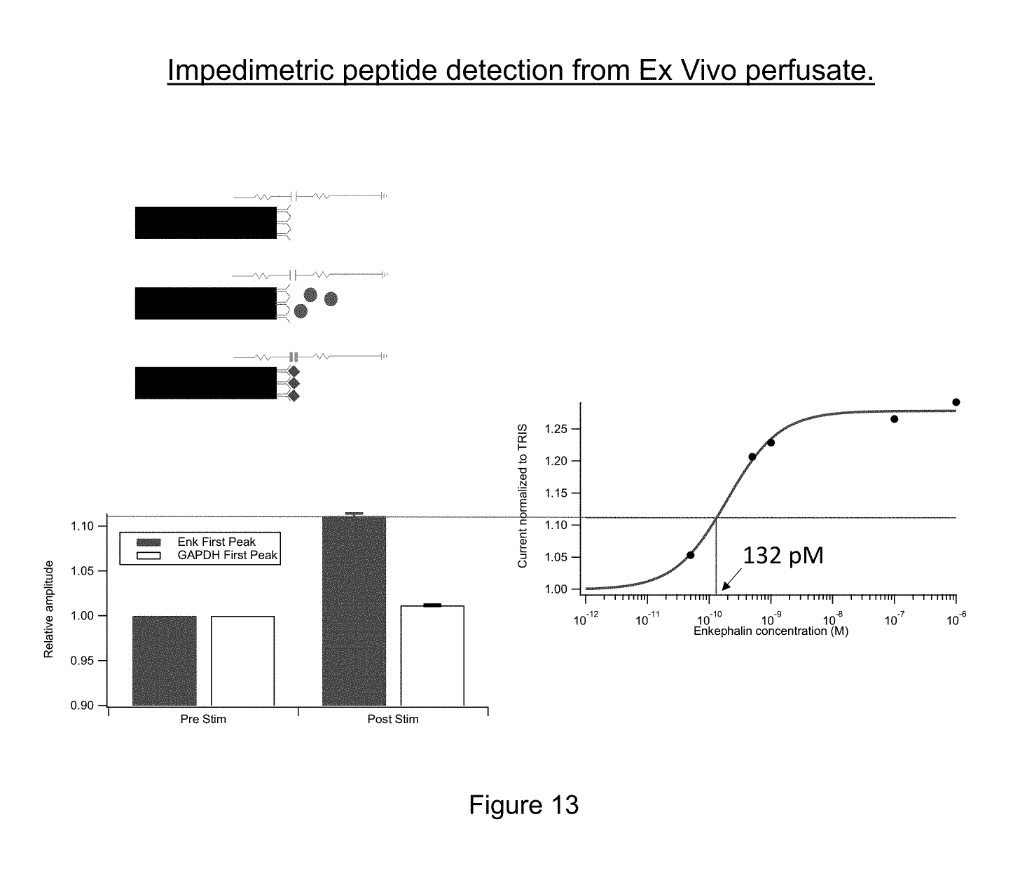

In order to determine whether specific chromaffin granule contents could be detected in intact tissue, carbon fiber electrodes were functionalized by covalently linking anti-enkephalin antibodies to the distal tip. Sample recordings are provided in FIG. 11 to demonstrate specificity of the probe for enkephalin versus non-specific BSA in solution. Then, paired electrodes were prepared for enkephalin (positive) and a non-secretory negative control peptide, GAPDH (in vitro calibration, FIG. 12). Signals for enkephalin (Enk) and GAPDH electrodes were acquired under a time-domain approach, including a two-step depolarization to avoid cross contamination by non-specific amperometric signals, and processed to measure the total charge input (charge (Q) capacitance(C) * voltage (V)) with a change in current amplitude serving an index of the change in capacitance. Resulting signals were specific for the Enk electrode as expected and a cross-calibration to a standard curve obtained under in vitro conditions revealed a signal indicating 132 picomolar pM Enk release, a value well within that expected and determined by other means (FIG. 13).

The disclosures of each and every patent, patent application, and publication cited herein are hereby incorporated herein by reference in their entirety. While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

What is claimed is:

1. A method for detecting a biochemical compound comprising the steps of:
inserting one or more electrodes in one or more locations selected from the group consisting of: the heart, neural structure, and peripheral blood vessel;
applying a voltage scan to the electrode; and
detecting a current indicative of the presence and abundance of the compound.

2. The method of claim 1, wherein the one or more electrodes are placed into the myocardium.

3. The method of claim 1, wherein the one or more electrodes are inserted via epicardial or vascular access.

4. The method of claim 1, wherein the compound is at least one catecholamine selected from the group consisting of norepinephrine and epinephrine.

5. The method of claim 1, wherein at least one electrode is an electrode selected from the group consisting of: wire electrodes, microwire electrodes, needle electrodes, plunge electrodes, penetrating electrodes, patch electrodes, single shank electrodes, 2D shank electrodes, 3D shank electrodes, and multi-electrode arrays.

6. The method of claim 1, wherein the voltage scan is a fast scanning cyclic voltammetry (FSCV) voltage scan.

7. The method of claim 6, wherein the FSCV voltage scan comprises a waveform selected from the group consisting of: a sawtooth pattern and sinusoidal pattern.

8. The method of claim 1, wherein the method comprises detecting the oxidation current of the compound.

9. The method of claim 1, wherein the method comprises constructing a voltammogram from the detected current, thereby identifying the compound.

10. The method of claim 9, comprising quantifying the abundance of the compound by plotting the peak current on a calibration curve.

11. The method of claim 1, wherein the one or more electrodes are placed in one or more locations selected from the group consisting of: a coronary sinus of the heart, a great vein of the heart, vena cava, left ventricle, aorta, right ventricle, right atria, left atria, pulmonary veins, pulmonary artery, stellate ganglia, dorsal root ganglia, epicardial fat pad, and pericardial fat pad.

12. The method of claim 1, wherein the presence and abundance of the biochemical compound is assessed in response to one or more cardiac stressors.

13. The method of claim 1, wherein a plurality of electrodes are placed at a plurality of locations within and around the heart to assess regional differences in the abundance of the biochemical compound.

14. A method for detecting a biochemical compound comprising the steps of:
inserting one or more electrodes in one or more locations selected from the group consisting of: the heart, neural structure, and peripheral blood vessel, wherein at least one electrode comprises a receptor molecule that specifically binds the biochemical compound; and
detecting a change in the capacitance of the electrode thereby indicating the presence of the biochemical compound.

15. The method of claim 14, wherein the biochemical compound is a protein or peptide that specifically binds to the receptor molecule.

16. The method of claim 14, wherein the level of the compound is detected in at least one ganglia selected from the group consisting of intrathoracic ganglia, stellate ganglia, autonomic ganglia, nodose ganglia, dorsal root ganglia and petrosal ganglia.

17. The method of claim 14, wherein one or more electrodes are placed in a peripheral artery or peripheral vein.

18. A biochemical compound detection device, comprising:
a controller, comprising a potentiostat;
a reference electrode communicatively connected to the controller; and
a one or more measurement electrodes communicatively connected to the controller;
wherein the controller is configured to apply an electric potential across the reference electrode and the one or more measurement electrodes, and to measure the current passing through the one or more measurement electrodes over time; and
wherein the reference electrode and one or more measurement electrodes are configured to measure the presence and concentration of one or more biochemical compounds.

19. The biochemical compound detection device of claim 17, further comprising a ground electrode, wherein the controller is configured to apply an electric potential across the reference electrode and the ground electrode.

20. The biochemical compound detection device of claim 17, wherein at least one measurement electrode comprises a receptor molecule that specifically binds to a biochemical compound.

21. The biochemical compound detection device of claim 18, further comprising a semi-permeable membrane applied to a portion of an electrode selected from the group consisting of the reference electrode, the measurement electrode, and the ground electrode.

22. The biochemical compound detection device of claim 17, wherein at least one of the electrodes selected from the group consisting of the measurement electrode and the reference electrode are made of platinum.

23. The biochemical compound detection device of claim 17, wherein at least one of the electrodes selected from the group consisting of the measurement electrode and the reference electrode are made from carbon fiber.

24. The biochemical compound detection device of claim 17, wherein the controller further comprises a voltage clamp, configured to maintain a substantially constant voltage across two or more electrodes.
